(19) United States
(12) Patent Application Publication
(10) Pub. No.: US 2013/0072809 A1
(43) Pub. Date: Mar. 21, 2013

(54) Method And System For Analyzing An EEG Recording

(71) Applicants: Scott B. Wilson, Del Mar, CA (US); Nicolas Nierenberg, La Jolla, CA (US); Mark Scheuer, Wexford, PA (US)

(72) Inventors: Scott B. Wilson, Del Mar, CA (US); Nicolas Nierenberg, La Jolla, CA (US); Mark Scheuer, Wexford, PA (US)

(73) Assignee: PERSYST DEVELOPMENT CORPORATION, San Diego, CA (US)

(21) Appl. No.: 13/620,855

(22) Filed: Sep. 15, 2012

Related U.S. Application Data
(60) Provisional application No. 61/536,236, filed on Sep. 19, 2011.

(51) Int. Cl. A61B 5/0476; A61B 5/048

(52) U.S. Cl. 600/544

(57) ABSTRACT

A method and system for analyzing EEG data is disclosed herein. A processed EEG recording is analyzed to produce a parameter for the EEG. The EEG is analyzed to organize a plurality of detections by spike focus, to determine a relative frequency based on a count of detections by spike focus, to average a plurality of detections by spike focus, and the like.

### CROSS REFERENCE TO RELATED APPLICATION

The Present Application claims priority to U.S. Provisional Patent Application No. 61/536236, filed on Sep. 19, 2011, which is hereby incorporated by reference in its entirety.

### STATEMENT REGARDING FEDERALLY SPONSORED RESEARCH OR DEVELOPMENT

Not Applicable

### BACKGROUND OF THE INVENTION

1. Field of the Invention

The present invention generally relates to EEG recordings. More specifically, the present invention relates to analyzing an EEG recording.

2. Description of the Related Art

An electroencephalogram (“EEG”) is a diagnostic tool that measures and records the electrical activity of a person's brain in order to evaluate cerebral functions. Multiple electrodes are attached to a person's head and connected to a machine by wires. The machine amplifies the signals and records the electrical activity of a person's brain. The electrical activity is produced by the summation of neural activity across a plurality of neurons. These neurons generate small electric voltage fields. The aggregate of these electric voltage fields create an electrical reading which electrodes on the person's head are able to detect and record. An EEG is a superposition of multiple simpler signals. In a normal adult, the amplitude of an EEG signal typically ranges from 1 micro-Volt to 100 micro-Volts, and the EEG signal is approximately 10 to 20 milli-Volts when measured with subdural electrodes. The monitoring of the amplitude and temporal dynamics of the electrical signals provides information about the underlying neural activity and medical conditions of the person.

An EEG is performed to: diagnose epilepsy; verify problems with loss of consciousness or dementia; verify brain activity for a person in a coma; study sleep disorders, monitor brain activity during surgery, and additional physical problems.

Multiple electrodes (typically 17-21, however there are standard positions for at least 70) are attached to a person's head during an EEG. The electrodes are referenced by the position of the electrode in relation to a lobe or area of a person's brain. The references are as follows: F=frontal; Fp=frontopolar; T=temporal; C=central; P=parietal; O=occipital; and A=auricular (ear electrode). Numerals are used to further narrow the position and “z” points relate to electrode sites in the midline of a person's head. An electrocardiogram (“EKG”) may also appear on an EEG display.

The EEG records brain waves from different amplifiers using various combinations of electrodes called montages. Montages are generally created to provide a clear picture of the spatial distribution of the EEG across the cortex. A montage is an electrical map obtained from a spatial array of recording electrodes and preferably refers to a particular combination of electrodes examined at a particular point in time.

In a bipolar montage, consecutive pairs of electrodes are linked by connecting the electrode input 2 of one channel to input 1 of the subsequent channel, so that adjacent channels have one electrode in common. The bipolar chains of electrodes may be connected going from front to back (longitudinal) or from left to right (transverse). In a bipolar montage signals between two active electrode sites are compared resulting in the difference in activity recorded. Another type of montage is the referential montage or monopolar montage. In a referential montage, various electrodes are connected to input 1 of each amplifier and a reference electrode is connected to input 2 of each amplifier. In a reference montage, signals are collected at an active electrode site and compared to a common reference electrode.

Reference montages are good for determining the true amplitude and morphology of a waveform. For temporal electrodes, CZ is usually a good scalp reference.

Being able to locate the origin of electrical activity (“localization”) is critical to being able to analyze the EEG. Localization of normal or abnormal brain waves in bipolar montages is usually accomplished by identifying “phase reversal,” a deflection of the two channels within a chain pointing to opposite directions. In a referential montage, all channels may show deflections in the same direction. If the electrical activity at the active electrodes is positive when compared to the activity at the reference electrode, the deflection will be downward. Electrodes where the electrical activity is the same as at the reference electrode will not show any deflection. In general, the electrode with the largest upward deflection represents the maximum negative activity in a referential montage.

Some patterns indicate a tendency toward seizures in a person. A physician may refer to these waves as “epileptiform abnormalities” or “epilepsy waves.” These include spikes, sharp waves, and spike-and-wave discharges. Spikes and sharp waves in a specific area of the brain, such as the left temporal lobe, indicate that partial seizures might possibly come from that area. Primary generalized epilepsy, on the other hand, is suggested by spike-and-wave discharges that are widely spread over both hemispheres of the brain, especially if they begin in both hemispheres at the same time.

There are several types of brain waves: alpha waves, beta waves, delta wave, theta waves and gamma waves. Alpha waves have a frequency of 8 to 12 Hertz (“Hz”). Alpha waves are normally found when a person is relaxed or in a waking state when a person's eyes are closed but the person is mentally alert. Alpha waves cease when a person's eyes are open or the person is concentrating. Beta waves have a frequency of 13 Hz to 30 Hz. Beta waves are normally found when a person is alert, thinking, agitated, or has taken high doses of certain medicines. Delta waves have a frequency of less than 3 Hz. Delta waves are normally found only when a person is asleep (non-REM or dreamless sleep) or the person is a young child. Theta waves have a frequency of 4 Hz to 7 Hz. Theta waves are normally found only when the person is asleep (dream or REM sleep) or the person is a young child. Gamma waves have a frequency of 30 Hz to 100 Hz. Gamma waves are normally found during higher mental activity and motor functions.

The following definitions are used herein.

“Amplitude” refers to the vertical distance measured from the trough to the maximal peak (negative or positive). It expresses information about the size of the neuron population and its activation synchrony during the component generation.

The term “analogue to digital conversion” refers to when an analogue signal is converted into a digital signal which can then be stored in a computer for further processing. Analogue signals are “real world” signals (e.g., physiological signals such as electroencephalogram, electrocardiogram or electrooculogram). In order for them to be stored and manipulated by a computer, these signals must be converted into a discrete digital form the computer can understand.

“Artifacts” are electrical signals detected along the scalp by an EEG, but that originate from non-cerebral origin. There are patient related artifacts (e.g., movement, sweating, ECG, eye movements) and technical artifacts (50/60 Hz artifact, cable movements, electrode paste-related).

The term “differential amplifier” refers to the key to electrophysiological equipment. It magnifies the difference between two inputs (one amplifier per pair of electrodes).

“Duration” is the time interval from the beginning of the voltage change to its return to the baseline. It is also a measurement of the synchronous activation of neurons involved in the component generation.

“Electrode” refers to a conductor used to establish electrical contact with a nonmetallic part of a circuit. EEG electrodes are small metal discs usually made of stainless steel, tin, gold or silver covered with a silver chloride coating. They are placed on the scalp in special positions.

“Electrode gel” acts as a malleable extension of the electrode, so that the movement of the electrodes leads is less likely to produce artifacts. The gel maximizes skin contact and allows for a low-resistance recording through the skin.

The term “electrode positioning” (10/20 system) refers to the standardized placement of scalp electrodes for a classical EEG recording. The essence of this system is the distance in percentages of the 10/20 range between Nasion-Inion and fixed points. These points are marked as the Frontal pole (Fp), Central (C), Parietal (P), occipital (O), and Temporal (T). The midline electrodes are marked with a subscript z, which stands for zero. The odd numbers are used as subscript for points over the left hemisphere, and even numbers over the right

“Electroencephalogram” or “EEG” refers to the tracing of brain waves, by recording the electrical activity of the brain from the scalp, made by an electroencephalograph.

“Electroencephalograph” refers to an apparatus for detecting and recording brain waves (also called encephalograph).

“Epileptiform” refers to resembling that of epilepsy.

“Filtering” refers to a process that removes unwanted frequencies from a signal.

“Filters” are devices that alter the frequency composition of the signal.

“Montage” means the placement of the electrodes. The EEG can be monitored with either a bipolar montage or a referential one. Bipolar means that there are two electrodes per one channel, so there is a reference electrode for each channel. The referential montage means that there is a common reference electrode for all the channels.

“Morphology” refers to the shape of the waveform. The shape of a wave or an EEG pattern is determined by the frequencies that combine to make up the waveform and by their phase and voltage relationships. Wave patterns can be described as being: “Monomorphic”. Distinct EEG activity appearing to be composed of one dominant activity. “Polymorphic”. distinct EEG activity composed of multiple frequencies that combine to form a complex waveform. “Sinusoidal”. Waves resembling sine waves. Monomorphic activity usually is sinusoidal. “Transient”. An isolated wave or pattern that is distinctly different from background activity.

“Spike” refers to a transient with a pointed peak and a duration from 20 to under 70 msec.

The term “sharp wave” refers to a transient with a pointed peak and duration of 70-200 msec.

The term “neural network algorithms” refers to algorithms that identify sharp transients that have a high probability of being epileptiform abnormalities.

“Noise” refers to any unwanted signal that modifies the desired signal. It can have multiple sources.

“Periodicity” refers to the distribution of patterns or elements in time (e.g., the appearance of a particular EEG activity at more or less regular intervals). The activity may be generalized, focal or lateralized.

An EEG epoch is an amplitude of a EEG signal as a function of time and frequency.

Various techniques have been developed to present the EEG data to a physician or technician. However, these techniques are still lacking If the raw EEG report is presented to a physician or technician, then artifacts typically render the EEG report incapable of distinguishing brain activity such as a seizure from artifacts. Despite the use of artifact reduction algorithms, the failure to accurately distinguish true physiological rhythmicity from the artifacts is a serious shortcoming of current software systems and requires an expert assessment.

An EEG report produces tremendous amounts of information about a person's brain activity. However, there is a need to quickly and easily interpret that information in order to properly analyze the brain activity of a person.

### BRIEF SUMMARY OF THE INVENTION

One aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes analyzing the processed EEG recording to produce a parameter for the EEG.

Another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes organizing a plurality of detections by spike focus.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes determining a relative frequency based on a count of detections by spike focus.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes creating a back-to-back view of spike detections organized by spike focus.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes selecting an EEG clip of a spike focus to view an extended portion of the EEG for context.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes averaging a plurality of detections by spike focus on a summary.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes moving from an average of a plurality of detections by spike focus to an individual detection.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes marking a plurality of spike averages and a plurality of individual detections at spike focus.

Yet another aspect of the present invention is a method for analyzing an EEG recording. The method includes generating an EEG recording from a machine comprising a plurality of electrodes, an amplifier and processor. The method also includes processing the EEG to create a processed EEG recording for analysis. The method also includes determining which of a plurality of spike detections to include in a grouping, an averaging or a final analysis by changing a sensitivity of the EEG to view a detection.

Yet another aspect of the present invention is a method for analyzing an EEG recording. The method includes generating an EEG recording from a machine comprising a plurality of electrodes, an amplifier and processor. The method also includes processing the EEG recording with a plurality of neural network algorithms to create a processed EEG recording for analysis. The method also includes analyzing the processed EEG recording to produce a parameter for the EEG.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to analyze a processed EEG recording to produce a parameter for the EEG.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to organize a plurality of detections of the processed EEG recording by spike focus.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to determine a relative frequency based on a count of detections by spike focus.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to create a back-to-back view of spike detections organized by spike focus.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to select an EEG clip of a spike focus of the processed EEG recording to view an extended portion of the EEG for context.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to average a plurality of detections of the processed EEG recording by spike focus on a summary.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to move from an average of a plurality of detections of the processed EEG recording by spike focus to an individual detection.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to mark a plurality of spike averages of the processed EEG recording and a plurality of individual detections of the processed EEG recording at spike focus.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to determine which of a plurality of detections of the processed EEG recording to include in a final analysis by changing a sensitivity of the EEG to view a detection.

Yet another aspect of the present invention is a system for analyzing an EEG recording. The system includes a plurality of electrodes, at least one amplifier, a processor and a display. The plurality of electrodes generates a plurality of EEG signals. The at least one amplifier is connected to each of the plurality of electrodes by a plurality of wires to amplify each of the plurality of EEG signals. The processor is connected to the amplifier to generate an EEG recording from the plurality of EEG signals. The display is connected to the processor for displaying an EEG recording. The processor is configured to process the EEG recording with a plurality of neural network algorithms to create a processed EEG recording.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a block diagram of a system for analyzing an EEG recording.

FIG. 2 is an illustration of an analyzed EEG recording.

FIG. 3 is an illustration of a raw detection display of an analyzed EEG recording.

FIG. 4 is an illustration of an expanded detection view display of an analyzed EEG recording.

FIG. 5 is an illustration of a final report display of an analyzed EEG recording.

FIG. 6 is a flow chart of a general method for analyzing an EEG recording.

FIG. 7 is an illustration of an EEG recording for a normal awake patient.

FIG. 7A is an illustration of an analyzed EEG recording for a generalized spike EEG.

FIG. 7B is an illustration of an analyzed EEG recording for a focal spike EEG.

FIG. 8 is a block diagram of a system for analyzing an EEG recording.

FIG. 9 is a flow chart of a general method for analyzing an EEG recording.

FIG. 10 is a flow chart of a specific method for analyzing an EEG recording.

FIG. 11 is a flow chart of a specific method for analyzing an EEG recording.

FIG. 12 is a map representing the international 10-20 electrode system for electrode placement for an EEG.

FIG. 13 is a detailed map representing the intermediate 10% electrode positions, as standardized by the American Electroencephalographic Society, for electrode placement for an EEG.

### DETAILED DESCRIPTION OF THE INVENTION

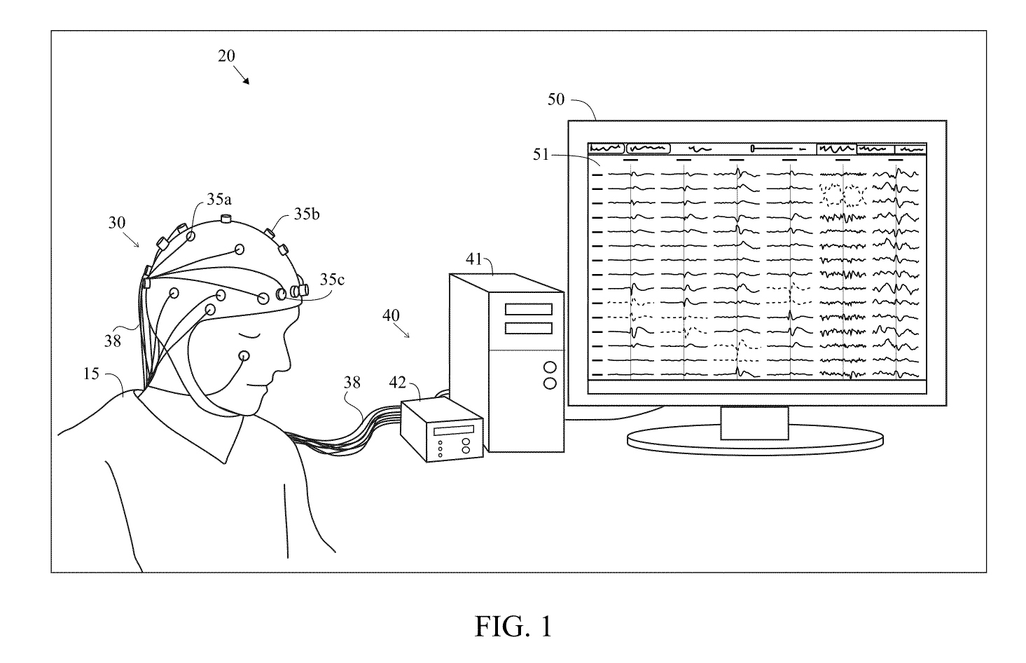

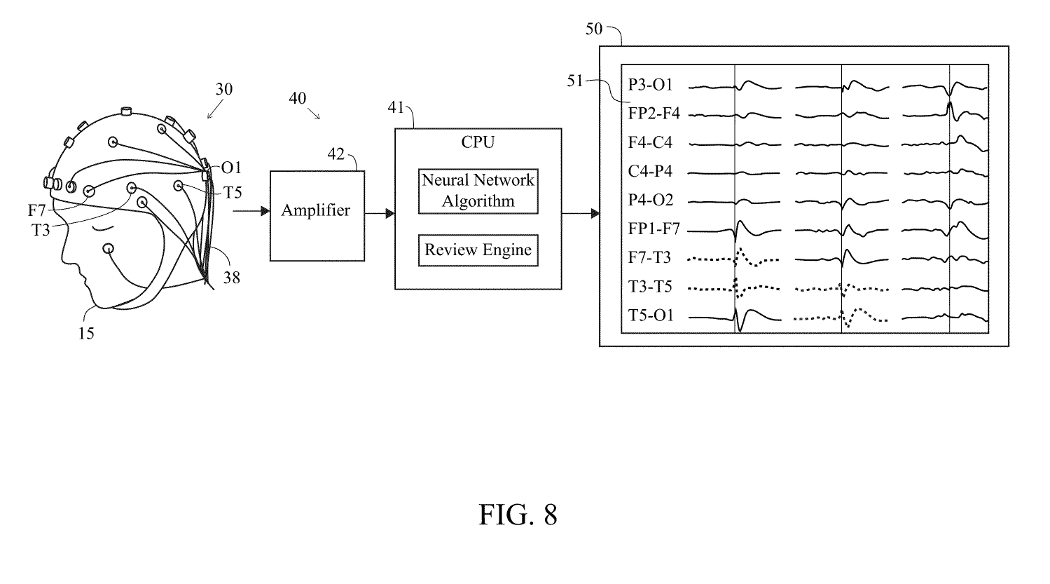

As shown in FIG. 1, an EEG system is generally designated 20. The system preferably includes a patient component 30, an EEG machine component 40 and a display component 50. The patient component 30 includes a plurality of electrodes 35a, 35b, 35c attached to the patient 15 and wired by cables 38 to the EEG machine component 40. The EEG machine component 40 preferably comprises a CPU 41 and an amplifier component 42. The EEG machine component 40 is connected to the display component 50 for display of the combined EEG reports, and for switching from a processed EEG report to the combined EEG reports, or from the processed EEG report to an original EEG report. As shown in FIG. 8, the EEG machine component 40 preferably includes a review engine and neural network algorithms. The machine component also preferably comprises a memory, a memory controller, a microprocessor, a DRAM, and an Input/Output. Those skilled in the pertinent art will recognize that the machine component 40 may include other components without departing from the scope and spirit of the present invention. The EEG recordings are then processed using neural network algorithms to generate a processed EEG recording which is analyzed for display.

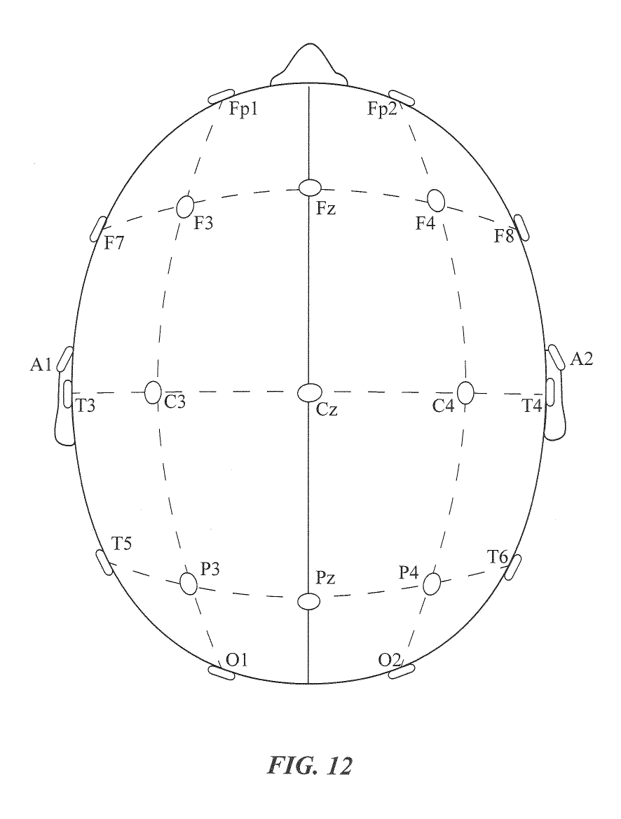

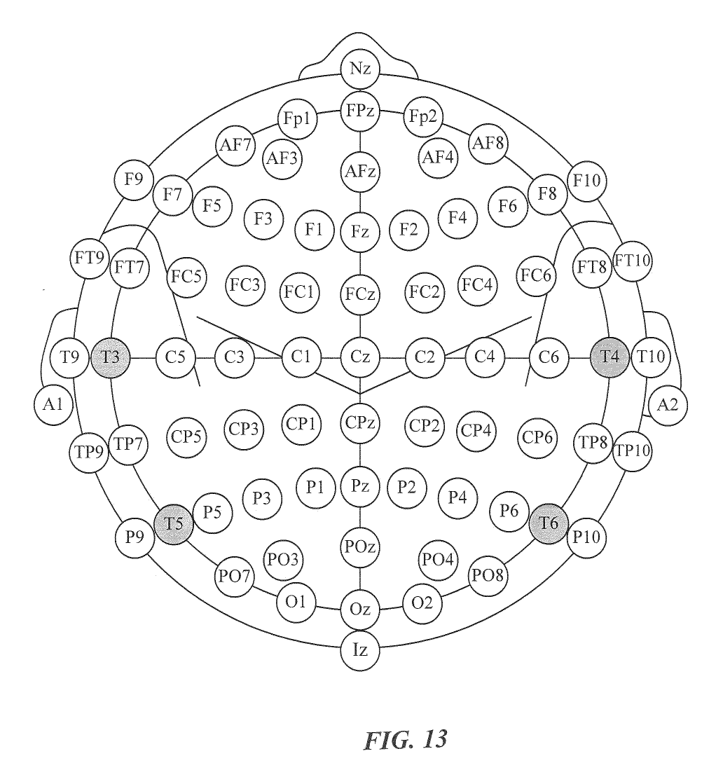

A patient has a plurality of electrodes attached to the patient's head with wires from the electrodes connected to an amplifier for amplifying the signal to a processor which is used to analyze the signals from the electrodes and create an EEG recording. The brain produces different signals at different points on a patient's head. Multiple electrodes are positioned on a patient's head as shown in FIGS. 12 and 13. For example Fp1 on FIG. 12 is represented in channel FP1-F7 on FIG. 8. The number of electrodes determines the number of channels for an EEG. A greater number of channels produces a more detailed representation of a patient's brain activity. Preferably, each amplifier of an EEG machine component 40 corresponds to two electrodes attached to a patient's head. The output from an EEG machine component is the difference in electrical activity detected by the two electrodes. The placement of each electrode is critical for an EEG report since the closer electrode pairs are to each other, the less difference in the brainwaves that are recorded by the EEG machine component. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is analyzed for display.

Algorithms for removing artifact from EEG typically use Blind Source Separation (BSS) algorithms like CCA (canonical correlation analysis) and ICA (Independent Component Analysis) to transform the signals from a set of channels into a set of component waves or “sources.” The sources that are judged as containing artifact are removed and the rest of the sources are reassembled into the channel set.

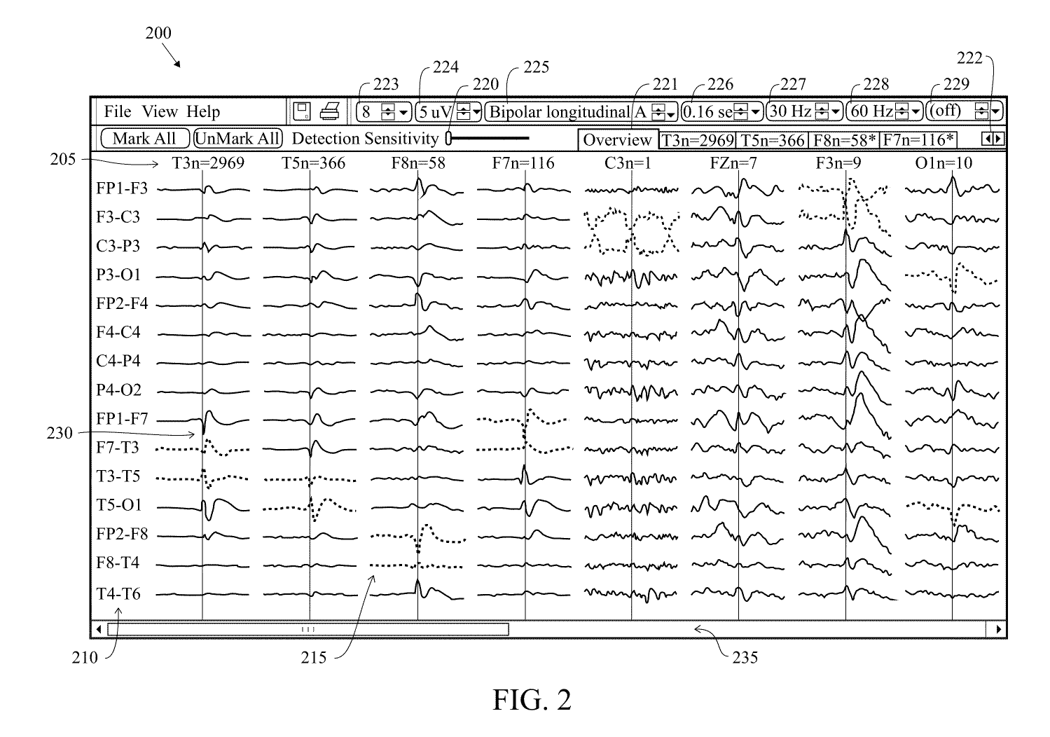

FIGS. 2-5 illustrate analyzed EEG recordings. As shown in FIG. 2, a display of an analyzed EEG recording on a computer screen is designated 200. Reference 205 designates the electrode foci (T3) and the number of detections (2969) selected at this sensitivity. The montage bar is designated 210 and allows for montage controls. Reference 215 shows a primary electrode detection focus. The detection sensitivity slider is designated 220 and allows an operator to select the sensitivity for display. Dragging the slider to the right dynamically increases the detection sensitivity thereby yielding more true positives but also more false positives. Less sensitivity shows less spikes. The group tab is designated 221, and the tab is used to select the detection group displayed in the main window. The following types of tabs are available: Overview which is detection averages arranged by electrode focus, showing averages of all detections at chosen detection sensitivity; Individual Electrode Foci, for example T3, T5; Final Report which is spike averages of hand chosen detections, sorted by electrode focus. The number of detections is shown at each focus for the chosen sensitivity. The navigation tabs are designated 222, which allow for navigation to other tabs not currently in view on the window. The spike detections per page tab is designated 223 and allows for a number of detections that yields about 30 mm per each one second spike detection event. The EEG voltage amplitude selector is designated 224. The montage selector tab is 225, the LFF tab is 226, the HFF tab is 227, the notch tab is 228, and the customer filter tab is 229. An operator can jump to a group's constituent spikes by clicking on the group such as at point 230. The page forward tab is 235.

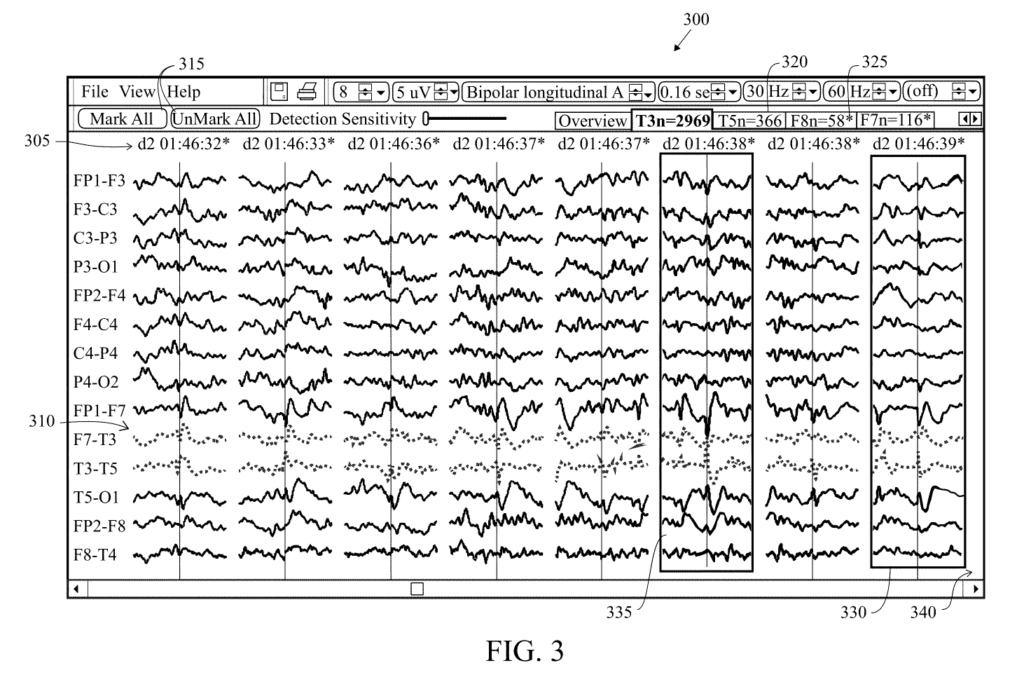

As shown in FIG. 3, a display showing raw detections at T3 of an analyzed EEG recording on a computer screen is designated 300. A time of detection is designated 305. Electrodes involved in the detection are typically highlighted, and shown as reference 310. The mark or unmark tab is 315, which allows for marked detections to appear in the final report. The navigate tab 320 allows for navigation between detection foci. As shown at 325, detections that have already been viewed are marked with an asterisk. A hand marked detection 330 places a box around the detection. EEG centered on the spike detection is shown at 335. Tab 340 allows for movement to the next page of detections.

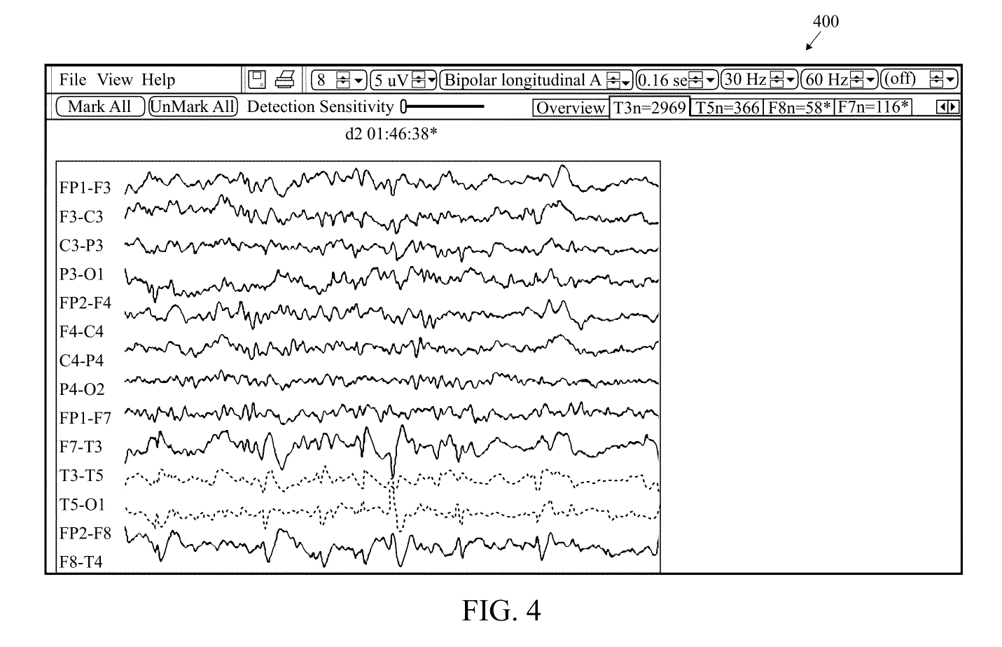

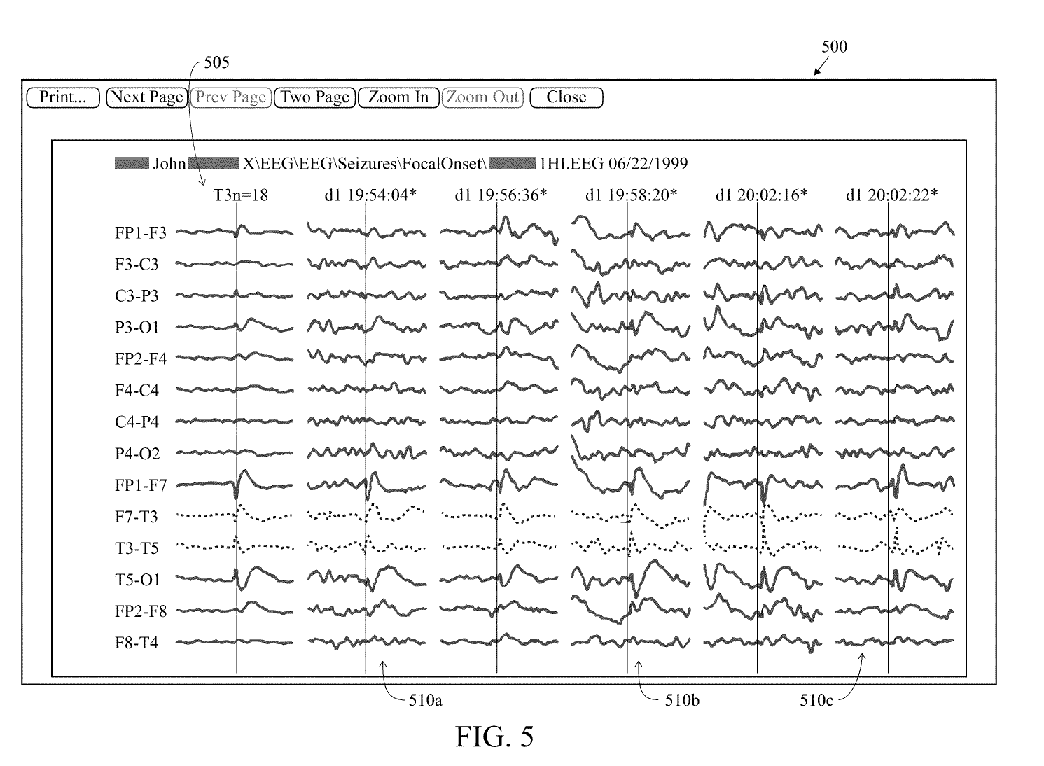

As shown in FIG. 4, a display 400 of an analyzed EEG recording on a computer screen shows an expanded detection view. As shown in FIG. 5, a final report display of an analyzed EEG recording on a computer screen is designated 500. An average of user selected spikes with T3 voltage maximum is shown at reference 505. 510a, 510b and 510c are individual constituent user selected spikes.

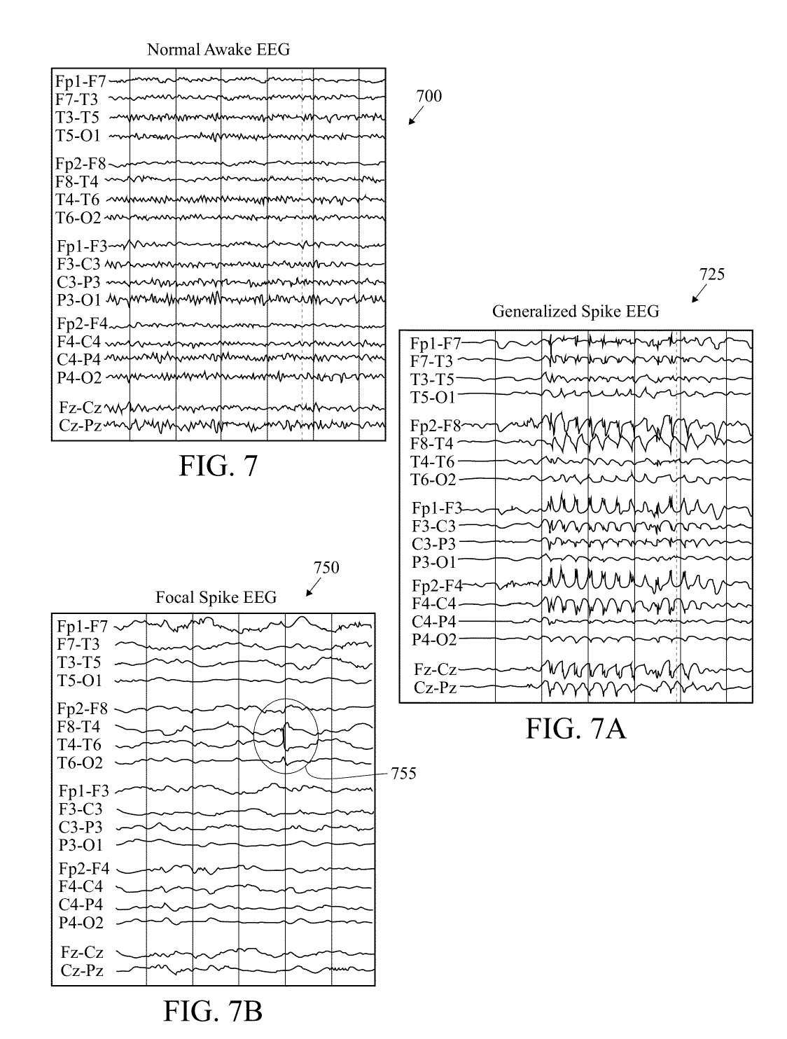

An EEG 700 for a normal awake patient is shown in FIG. 7. An EEG 725 having generalized spikes is shown in FIG. 7A. An EEG 750 having a focal spike is shown in FIG. 7B.

In use for analyzing an EEG recording, a technician or physician activates the review program, and after a few seconds the review program opens. The overview window is initially presented. The overview depicts averages from the various spike foci detected by a spike detection mechanism. To create these overview averages the spike detections are sorted by detection foci (electrode) and then all detections at a particular focus are mathematically averaged. For example, the first column of EEG represents an average of 2969 events that had their maximum point of detection at the T3 electrode. The columns of the EEG are preferably separated from other columns by a thin band of white. Each EEG column represents a distinct group average. The primary electrode focal point of each average, and the number of detection events incorporated into each average, are shown above the columns of EEG. Channels including the detection focal point electrode are highlighted red. As with evoked potentials, averaging multiple detections results in an increase in the signal-to-noise ratio and makes it easier to delineate the field of distribution of epileptiform abnormalities.

The sensitivity of the SpikeDetector output can be dynamically adjusted during the review process. This is done by using the detection sensitivity slider, which is labeled. When Easy SpikeReview is initially opened, the detection sensitivity slider is set to the far left position. In this position the SpikeDetector neural network algorithms identify sharp transients that have a high probability of being epileptiform abnormalities: these are events the detector assigned a high probability of being a real epileptiform abnormality. The rate of false positive detections at this setting is lowest. Thus, the ratio of true epileptiform signal to false positive noise is highest at this setting. However, some spikes and sharp waves that are less well-formed may not be evident with the slider set at its lowest sensitivity. The detector's sensitivity can be quickly adjusted by dragging the slider towards the right so that it is more sensitive and thus more likely to identify less well-formed or lower amplitude transients. New groups may then appear in the overview display of spike averages. In concert with the increase in true spike detections, there is also an increase in false positive detections.

An example of changes in the number of detected events associated with moving the detection sensitivity adjustment slider from the left to the far right (see red arrows) is shown. Looking only at the T3 detections, the number of detected events increased from 2969 to 4528 (see yellow highlights).

In records with rare epileptiform abnormalities or those in which the SpikeDetector neural networks, when set to lowest sensitivity, do not recognize the epileptiform abnormalities well, switching to the highest setting on the detection sensitivity slider may allow visualization of real epileptiform abnormalities. In such cases, identifying the rare events often requires assessment of the individual raw detections. This is accomplished by either displaying all raw detections back-to-back following the spike averages on the overview page, or by reviewing the detections at each electrode location by progressively selecting the location tabs at the top of the EEG window (see below).

Clicking on any of the electrode location tabs at the top of the EEG window will display the raw (non-averaged) spike detections that arose from that particular electrode location. The individual detections are separated by a thin band of white, and the detection point is centered in a one second segment of EEG and indicated by a faint vertical gray line. Left double-clicking with the mouse on any individual detection will cause an expanded EEG view of that particular detection to appear. Left double-clicking on the expanded view will return the user to a display of back-to-back individual detections.

When viewing individual spike detections (accessed from the tabs above the EEG window), exemplar spikes can be hand-marked by left-clicking with the mouse on the desired example. A rectangle outlining the chosen spike will appear. Hand-marked detections will be included in the spike averages that appear in the FinalReport. These hand-marked events can also be displayed back-to-back, immediately following their averages in FinalReport, and can be printed for archival purposes or for evaluation by another reviewer.

Clicking on FinalReport at the top of the EEG window displays a summary of all hand-marked events. The initial default view shows the mathematical averages of the user-chosen hand-marked events, sorted by electrode focus. As explained, head voltage topograms and back-to-back individual user-selected events are displayed by selecting menu options or via right mouse click choices. Voltage topograms are only created when viewing the EEG in a referential montage.

Using the initial overview screen with the detection sensitivity slider set to the far left (lowest sensitivity), identify any clear-cut or probable epileptiform abnormalities and note their locations. Double-click on the epileptiform abnormalities to further verify their nature by viewing individual examples contributing to the averages. Mark exemplar individual events as desired (for a Final Report).

Slide the detection sensitivity slider to the far right (highest sensitivity) and reassess the overview screen to determine whether any other clear-cut or probable epileptiform abnormalities become evident.

If epileptiform abnormalities were discovered and verified via the overview display, proceed with further review of back-to-back raw detections, as indicated.

If no epileptiform abnormalities were evident on overview, methodically assess all raw detections by selecting the various location tabs and paging through the back-to-back event detections. If any real epileptiform abnormalities are discovered, click on these to mark them for inclusion in the Final Report and then continue a review.

Once a review is completed, go to Final Report. On the Final Report, choose whether to display only averages of epileptiform abnormalities or both averages and back-to-back hand-selected events. Print the Final Report examples, if desired.

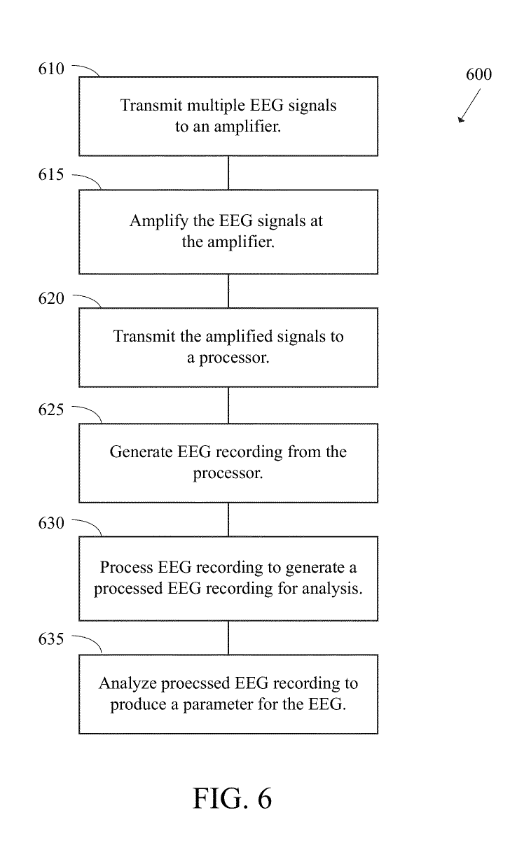

As shown in FIG. 6, a general method for analyzing an EEG recording is designated 600. At block 610, multiple EEG signals are transmitted to an amplifier. At block 615, the EEG signals are amplified by the amplifier. At block 620, the amplified signals are transmitted to a processor. At block 625, an EEG recording is generated by the processor. At block 630, the EEG recording is processed to generate a processed EEG recording for analysis. Processing preferably involves performing artifact reduction on the raw EEG recording. At block 635, the processed EEG recording is analyzed to produce a parameter for the EEG.

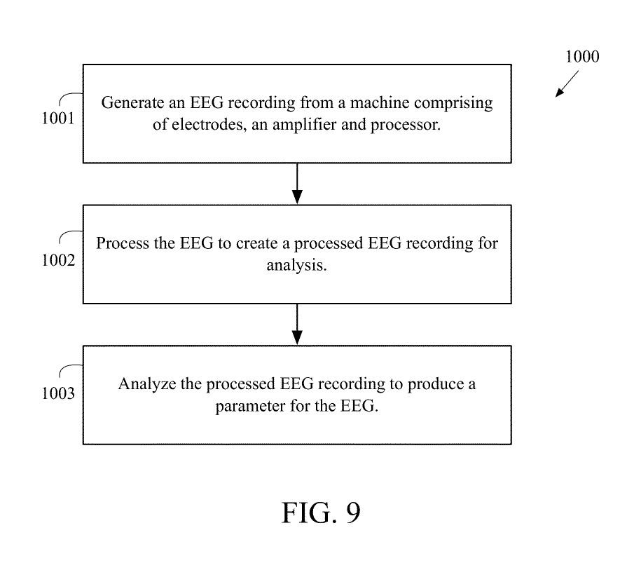

As shown in FIG. 9, a general method for analyzing an EEG recording is designated 1000. At block 1001, an EEG recording is generated from a machine comprising electrodes, an amplifier and a processor. At block 1002, the EEG recording is processed to create a processed EEG recording for analysis. At block 1003, the processed EEG recording is analyzed to produce a parameter for the EEG.

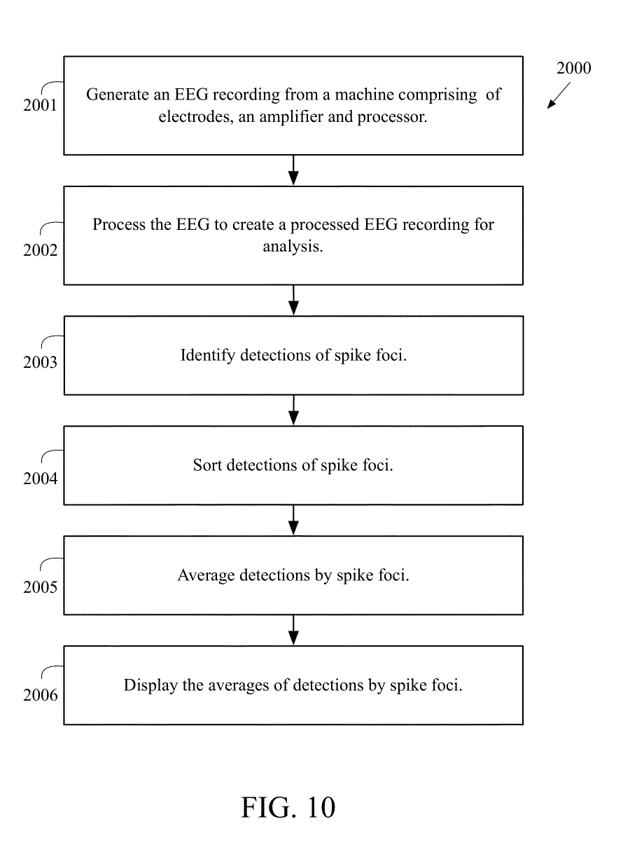

As shown in FIG. 10, a specific method for analyzing an EEG recording is designated 2000. At block 2001, an EEG recording is generated from a machine comprising electrodes, an amplifier and a processor. At block 2002, the EEG recording is processed to create a processed EEG recording for analysis. At block 2003, the detections by spike foci are identified in the processed EEG recording. At block 2004, the detections by spike foci are sorted, preferably by electrodes. At block 2005, the detections of spike foci are averaged. At block 2006, the averages of detections by spike foci are displayed for a physician or technician.

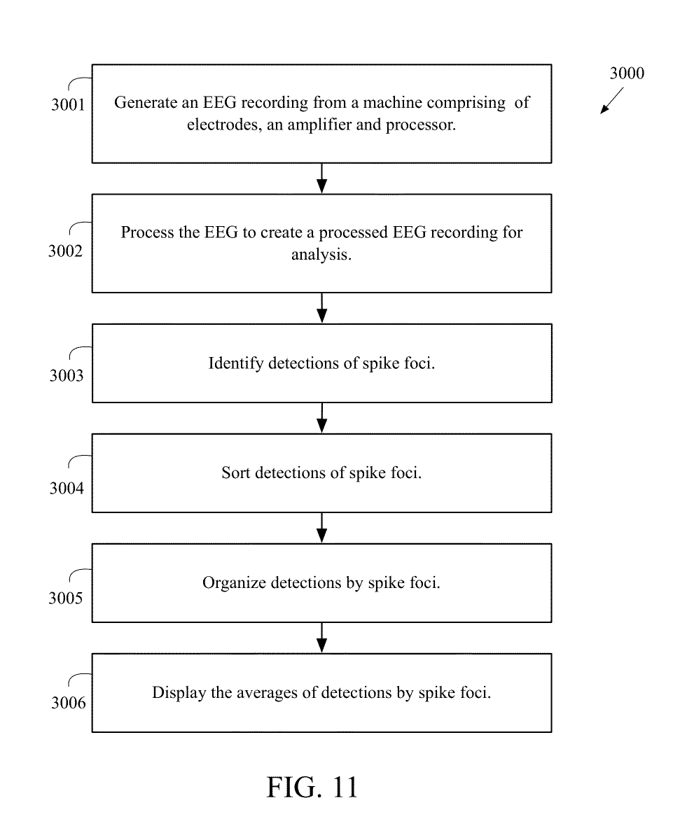

As shown in FIG. 11, a specific method for analyzing an EEG recording is designated 3000. At block 3001, an EEG recording is generated from a machine comprising electrodes, an amplifier and a processor. At block 3002, the EEG recording is processed to create a processed EEG recording for analysis. At block 3003, the detections by spike foci are identified in the processed EEG recording. At block 3004, the detections by spike foci are sorted, preferably by electrodes. At block 3005, the detections of spike foci are organized. At block 3006, the organization of detections by spike foci are displayed for a physician or technician.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention:

1. A method for analyzing an EEG recording, the method comprising:
generating an EEG recording from a machine comprising a plurality of electrodes, an amplifier and processor;
processing the EEG to create a processed EEG recording for analysis; and
analyzing the processed EEG recording to produce a parameter for the EEG.

2. The method according to claim 1 wherein analyzing the EEG comprises organizing a plurality of detections by spike focus

3. The method according to claim 1 wherein analyzing the EEG comprises determining a relative frequency based on a count of detections by spike focus.

4. The method according to claim 1 wherein analyzing the EEG comprises creating a back-to-back view of spike detections organized by spike focus.

5. The method according to claim 1 wherein analyzing the EEG comprises selecting an EEG clip of a spike focus to view an extended portion of the EEG for context.

6. The method according to claim 1 wherein analyzing the EEG comprises averaging a plurality of detections by spike focus on a summary.

7. The method according to claim 1 wherein analyzing the EEG comprises moving from an average of a plurality of detections by spike focus to an individual detection.

8. The method according to claim 1 wherein analyzing the EEG comprises marking a plurality of spike averages and a plurality of individual detections at spike focus.

9. The method according to claim 1 wherein analyzing the EEG comprises determining which of a plurality of spike detections to include in a grouping, an averaging or a final analysis by changing a sensitivity of the EEG to view a detection.

10. The method according to claim 1 wherein analyzing the EEG comprises changing the dynamic spike sensitivity to increase a plurality of candidate spikes.

11. The method according to claim 1 wherein analyzing the EEG comprises highlighting a plurality of channels at the spike focus.

12. The method according to claim 1 wherein processing the EEG recording comprises processing the EEG recording with a plurality of neural network algorithms.

13. A system for analyzing an EEG recording, the system comprising:
a plurality of electrodes for generating a plurality of EEG signals;
at least one amplifier connected to each of the plurality of electrodes by a plurality of wires to amplify each of the plurality of EEG signals;
a processor connected to the amplifier to generate an EEG recording from the plurality of EEG signals; and
a display connected to the processor for displaying an EEG recording;
wherein the processor is configured to analyze a processed EEG recording to produce a parameter for the EEG.

14. The system according to claim 13 wherein the processor is configured to organize a plurality of detections of the processed EEG recording by spike focus

15. The system according to claim 13 wherein the processor is configured to determining a relative frequency based on a count of detections by spike focus.

16. The system according to claim 13 wherein the processor is configured to create a back-to-back view of spike detections organized by spike focus.

17. The system according to claim 13 wherein the processor is configured to select an EEG clip of a spike focus of the processed EEG recording to view an extended portion of the EEG for context.

18. The system according to claim 13 wherein the processor is configured to average a plurality of detections of the processed EEG recording by spike focus on a summary.

19. The system according to claim 13 wherein the processor is configured to move from an average of a plurality of detections of the processed EEG recording by spike focus to an individual detection.

20. The system according to claim 13 wherein the processor is configured: to mark a plurality of spike averages of the processed EEG recording and a plurality of individual detections of the processed EEG recording at spike focus; to determine which of a plurality of detections of the processed EEG recording to include in a final analysis by changing a sensitivity of the EEG to view a detection; or to process the EEG recording with a plurality of neural network algorithms to create a processed EEG recording.
